(19) United States
(12) Patent Application Publication
(10) Pub. No.: US 2024/0173370 A1
(43) Pub. Date: May 30, 2024

(54) CONTROLLED RELEASE OF BACTERIOPHAGE TO TREAT IMPLANT INFECTIONS

(71) Applicant: The Board of Trustees of the Leland Stanford Junior University, Stanford, CA (US)

(72) Inventors: Ovijit CHAUDHURI, Stanford, CA (US); Paul L. BOLLYKY, Stanford, CA (US); Robert MANASHEROB, Stanford, CA (US); Yung-Hao LIN, Stanford, CA (US); Derek AMANATULLAH, Stanford, CA (US)

(73) Assignee: The Board of Trustees of the Leland Stanford Junior University, Stanford, CA (US)

(21) Appl. No.: 18/552,406

(22) PCT Filed: Mar. 25, 2022
(86) PCT No.: PCT/US2022/071351

§ 371 Date: Sep. 25, 2023

Related U.S. Application Data
(60) Provisional application No. 63/166,241, filed on Mar. 26, 2021.

(51) Int. Cl. A61K 35/76; A61K 47/10; A61K 47/61; A61K 47/69; A61P 31/04; C12N 7/00

(52) U.S. Cl. CPC A61K 35/76; A61K 47/10; A61K 47/61; A61K 47/6903; A61P 31/04; C12N 7/00

(57) ABSTRACT

Provided herein are hydrogels that include a plurality of bacteriophages located within and covalently bonded to the hydrogel interior. The hydrogel is engineered to facilitate a controlled sustained release of the connected bacteriophages, e.g., to or within the body of a patient suffering from a bacterial infection. Also provided are methods for forming the provided hydrogels, and for using the hydrogels to treat a patient suffering from a bacterial infection.

### CROSS-REFERENCE TO RELATED APPLICATIONS

The present application claims priority to U.S. Provisional Application No. 63/166,241 filed Mar. 26, 2021, the full disclosure of which is incorporated by reference in its entirety for all purposes.

### BACKGROUND

Orthopaedic joint infections affect nearly 11,000 patients per year and cost over $1.6 billion in 2020. During an orthopedic joint infection, bacteria bind to the implant and survive the administration of antibiotics by forming an antibiotic-tolerant biofilm. Therefore, treatment of orthopaedic joint infection requires surgical removal of biofilm-contaminated implants rather than administration of antibiotics alone. Current protocols call for months of intravenous antibiotics in between the removal of the infected hardware and the revision surgery, during which time the patient is bedridden. The morbidity, mortality, and economic cost of these procedures is massive. Moreover, the revised hardware is frequently re-infected, often due to persistent bacteria or antibiotic resistance.

Treatment with lytic bacteriophages (a.k.a., phages) is a promising approach to chronic antibiotic-resistant infections and biofilm-associated infections. Phages are viruses that infect bacteria, multiply, and then cause lysis and bacterial death. However, the current method of sustained, intravenous delivery of active bacteriophages over days to weeks is costly and inefficient. An alternative delivery method involves covalent bonding of bacteriophages to a hydrogel that is not engineered to undergo either hydrolytic or bacterial enzyme-mediated degradation. For example, covalent bonding of bacteriophages that undergo degradation mediated by matrix metalloproteinase (MMP) has been demonstrated with some success.

In view of this existing art, a need remains for a bacteriophage delivery vehicle capable of providing controlled sustained release of bacteriophage from the vehicle to a subject. The present disclosure addresses this need and provides associated and other advantages.

### BRIEF SUMMARY

In general, provided herein are functionalized hydrogels developed to deliver a pre-selected phage or a cocktail of phages, e.g., to the site of an orthopaedic hardware infection during primary surgery for prophylaxis as well as prior to or during revision surgery for treatment. Advantageously, this approach can prevent infection with common bacterial pathogens, block reinfection, and shorten the time of debilitation due to infection. Because the bacteria causing the infection typically can be readily isolated, residual infections can be treated, and new infections associated with implanted hardware can be prevented through use of bacteriophages with the right delivery vehicle. The provided biomaterials can also deliver bacteriophages to infected hardware sites over long timescales. For example, sustained release of active bacteriophages can be provided over a timescale of days to weeks. These developments can lead to a new paradigm in implant treatment, e.g., mitigation and prevention of orthopaedic implant infections.

In one aspect, the disclosure provides a hydrogel including a plurality of bacteriophages located within the hydrogel interior. At least a portion of the bacteriophages are connected to the hydrogel via one or more covalent bonds. In some embodiments, the one or more covalent bonds include one or more dynamic covalent bonds. The hydrogel is engineered to facilitate a sustained release of the connected bacteriophages.

In another aspect, the disclosure provides a method for treating a patient suffering from a bacterial infection. The method includes administering to the patient an effective amount of any of the hydrogels disclosed herein. In some embodiments, the bacterial infection includes an orthopedic implant infection.

### BRIEF DESCRIPTION OF THE DRAWINGS

FIG. 1 is an illustration of bacteriophage release from a hydrogel, mediated by either hydrogel degradation with phage tethered to the gel, release of a weak bond between phage and hydrogel (i.e. dynamic covalent crosslink), or a combination of both.

FIG. 2 is an illustration of various covalent bonding chemistries suitable for use with the provided hydrogels.

FIG. 3 is an illustration of specific exemplary approaches to tethering phage via dynamic crosslinks to an alginate hydrogel in accordance with a provided embodiment. Alginate is functionalized by hydrazine and crosslinked into a hydrogel with 4-arm PEG-aldehyde and 4-arm PEG-benzaldehyde. Phage is mixed in with hydrogel components prior to gelation, and crosslinks to hydrogel via imine bonds (lysine on phage bonding to aldehyde on PEG crosslinker) or hydrazone bonds (aldehyde on phage bonding to hydrazine group on alginate)

FIG. 4 presents an illustration and graph showing alginate oxidation and hydrogel degradation results for 14 days.

FIG. 5 is an image of results from a spot assay of a Pseudomonas aeruginosa AA245 lawn infected with serial dilutions of LCL3 bacteriophage samples encapsulated and released from 3 different alginate hydrogel formulations.

FIG. 6 is a graph of bacteriophage release from non-degradable, non-bonding alginate hydrogels in the first 72 hours.

FIG. 7 is an illustration of a workflow for in vitro phage release studies with bacterial lawn assays used to characterize phage release over time.

FIG. 8 is a graph of the cumulative release of LBL3 phage from a provided alginate hydrogel, mediated by release of dynamic covalent bonds between phage and hydrogel

FIG. 9 is a graph of the daily release of LBL3 phage from a provided alginate hydrogel, mediated by release of dynamic covalent bonds between phage and hydrogel

FIG. 10 is an illustration of use of a provided hydrogel to treat periprosthetic joint infection. Bacteria from an implant infection are identified and used to select a cocktail of lytic bacteriophages. Then the bacteriophage cocktail is delivered to the infection via sustained local release from a hydrogel.

FIG. 11 presents an image of patient with a wound infected by Pseudomonas. (Left inset) Also shown is a transmission electron micrograph image of DMS3 phage that can target and kill Pseudomonas. (Right) Also shown is an illustration of a strategy to use a provided hydrogel to release bacteriophage locally in a sustained manner to treat the wound infection

### DETAILED DESCRIPTION

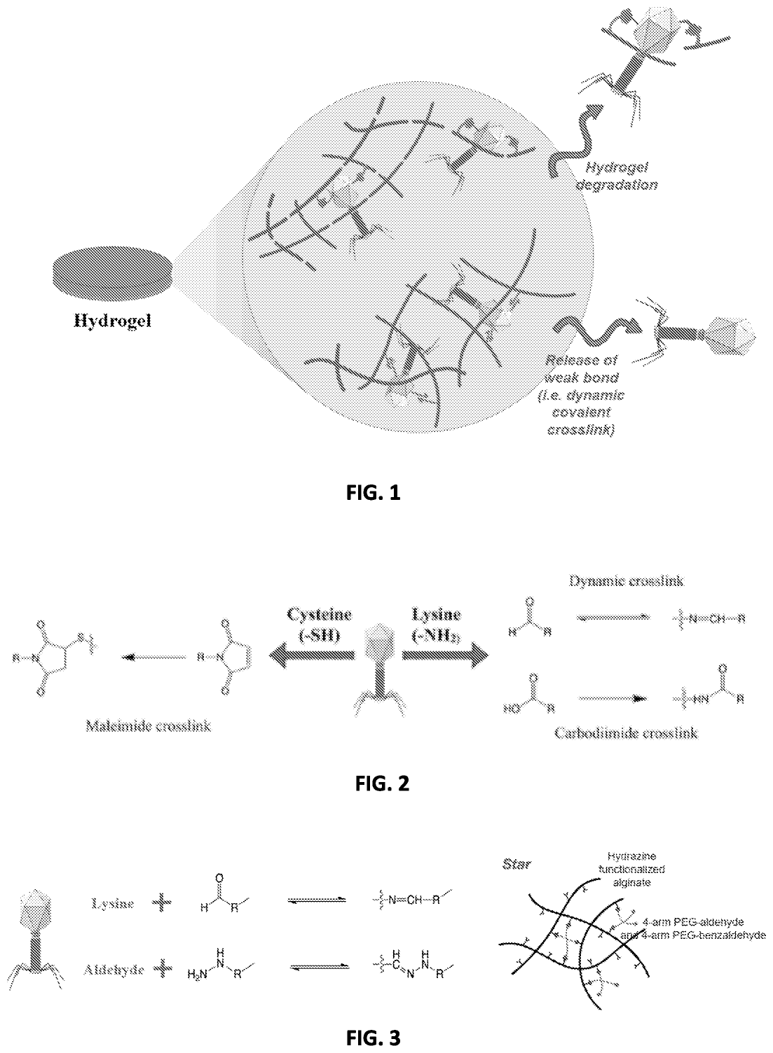

The inventors have surprisingly developed materials and methods involving the tunable release of bacteriophages from biomaterial carriers. Alginate hydrogels, known for their compatibility with various applications in regenerative medicine, have been found to be useful as biomaterial carriers for bacteriophages that allow controlled release (FIG. 1). The pore size of the hydrogel is on the order of 10 nm, a size sufficient to allow bacteriophage release from the gels over a timescale of hours. To delay and control bacteriophage release, bacteriophage can be bound to the alginate through various dynamic covalent or covalent crosslink chemistries.

Bacteriophage release can be facilitated through various complementary approaches. Unbinding, e.g., cleavage, of dynamic covalent crosslinks between the bacteriophage and hydrogel, followed by bacteriophage diffusion out of the hydrogel, provides one such mechanism. The timescale of unbinding can be controlled by using different chemistries for the covalent bond, including imine formation between lysine of bacteriophage capsid and aldehyde-functionalized alginate, carbodiimide crosslink between lysine and carboxyl moieties on alginate, or crosslink between cysteine and maleimide-functionalized alginate (FIG. 2).

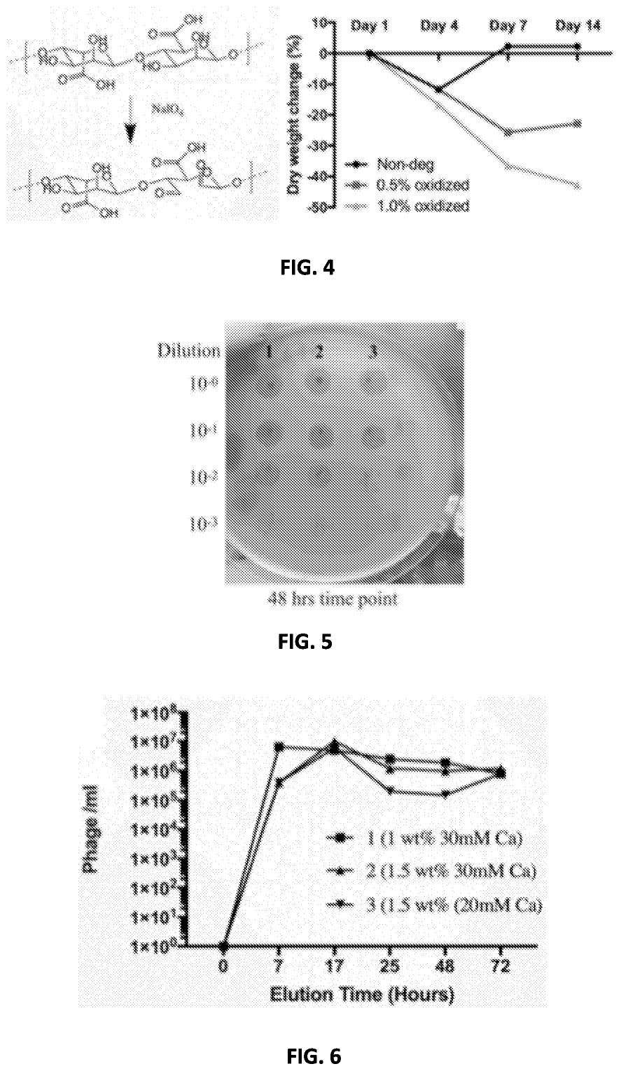

Further, engineered degradation of the hydrogel itself enables tuning of bacteriophage release. By controlling the degree of oxidation of alginate, hydrogel degradation can be varied from a timescale of hours to many weeks (FIG. 4). The timescale of delivery using these approaches can be experimentally characterized using, for example, a bacterial lawn assay (FIG. 5). In some embodiments of these assays, hydrogels containing bacteriophage are cultured in cell culture media in well plates, and the media is exchanged every day, with the removed media stored for analysis. The removed media is subsequently introduced to bacterial lawns, with the rate of bacterial growth inversely related to the bacteriophage activity. Release curves are then generated showing the cumulative release of active bacteriophage for various gel formulations. With these approaches, it is possible to tune release of bacteriophage from over hours to up to a week. In some embodiments, most of the bacteriophages release in 24 hours (FIG. 6). In other embodiments, covalent bonding of bacteriophages to alginate polymer chains and controlled release by hydrogel degradation extend the bacteriophage release to, e.g., 2 days, 3 days, 4 days, 5 days, 6 days, or one week. Bacteriophage releases of longer than one week are also contemplated.

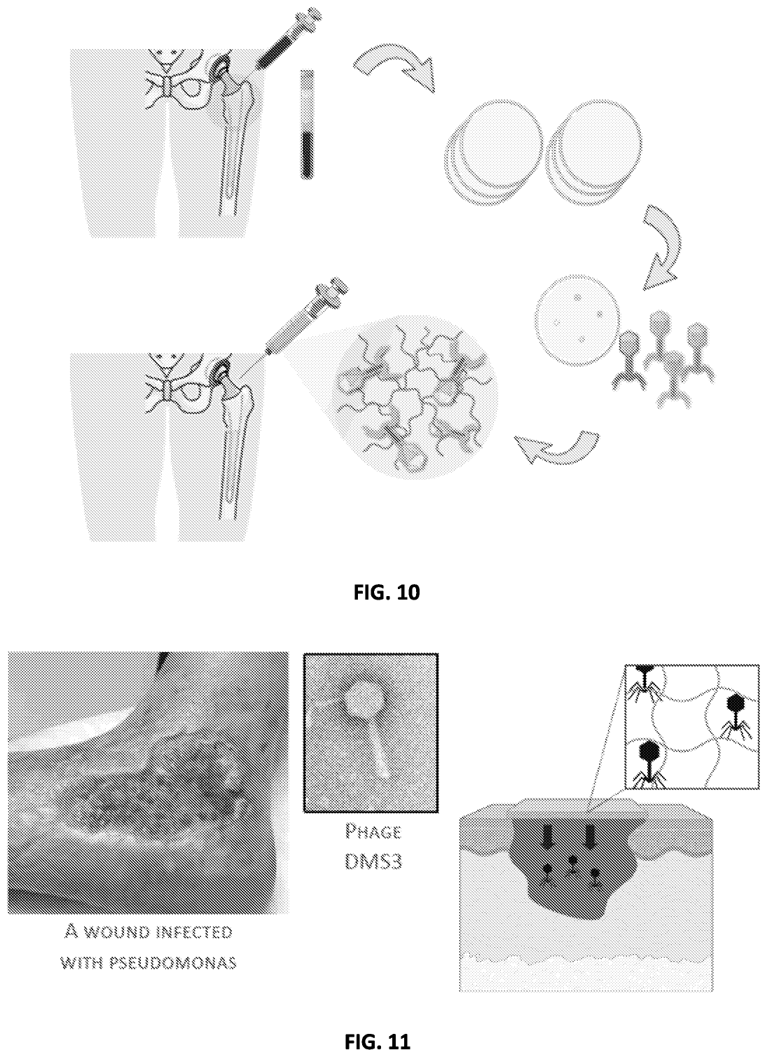

The provided device comprises three components: a hydrogel, a means for chemically bonding the bacteriophages to the hydrogel, and a mechanism enabling sustained release of the bacteriophage over long times. The device can be deployed locally (e.g., through injection or implantation) to treat bacterial infections therapeutically (FIGS. 10 and 11).

The hydrogel of the device can by formed, for example and without limitation, from one or more biopolymers such as alginate, chitosan, hyaluronic acid, collagen, gelatin or the synthetic polymers poly(ethylene glycol) (PEG), polyacrylamide (PAM), poly(hydroxyalkyl methacrylate) (PHEMA), poly(N-isopropylacrylamide) (PNIPAAm), poly(vinyl alcohol) (PVA), and polyesters such as poly(caprolactone) (PCL), poly(lactide) (PLA), or poly(lactic-co-glycolic acid) (PLGA). Any suitable crosslinking methods for the hydrogels can be used including one or more of electrostatic interaction (alginate/calcium), thermogelling (PEG/polyester copolymers), carbodiimide chemistry (polymer-NHS+polymer-NH₂), maleimide-sulfhydryl chemistry (polymer-maleimide-+polymer-cysteine), Diels-Alder chemistry (polymer-norbornene+polymer-tetrazine), thiol-ene chemistry (polymer-norbornene+polymer-cysteine), dynamic covalent crosslinks such as imine (polymer-NH₂+polymer-aldehyde), hydrazine (polymer-hydrazine+polymer-aldehyde), oxime (polymer-aldehyde+polymer-hydroxylamine) or boric acid and diols (polymer-diol+boric acid).

In some embodiments, the biopolymer, e.g., alginate, selected for forming the provided hydrogel is one having a low average molecular weight. For some applications, the molecular weight average of the biopolymer can be selected to provide desired phage release kinetics, improved biocompatibility, and other associated benefits. The molecular weight average of the biopolymer can be, for example, between 10 kDa and 80 kDa, e.g., between 10 kDa and 35 kDa, between 12 kDa and 43 kDa, between 15 kDa and 53 kDa, between 19 kDa and 65 kDa, or between 23 kDa and 80 kDa. In terms of upper limits, the biopolymer molecular weight average can be less than 80 kDa, e.g., less than 65 kDa, less than 53 kDa, less than 43 kDa, less than 35 kDa, less than 28 kDa, less than 23 kDa, less than 19 kDa, less than 15 kDa, or less than 12 kDa. In terms of lower limits, the biopolymer molecular weight average can be greater than 10 kDa, e.g., greater than 12 kDa, greater than 15 kDa, greater than 19 kDa, greater than 23 kDa, greater than 28 kDa, greater than 35 kDa, greater than 43 kDa, greater than 53 kDa, or greater than 65 kDa. Higher molecular weight averages. e.g., greater than 80 kDa, and lower molecular weight averages, e.g., less than 10 kDa, are also contemplated.

Chemistries for covalently bonding bacteriophages to the hydrogel can be covalent or dynamic covalent. Covalent bonding chemistries include, but are not limited to, carbodiimide chemistry (polymer-NHS, phage-NH₂), maleimide chemistry (polymer-maleimide, phage-SH), and Click chemistry, specifically alkyne-azide cycloaddition (polymer-azide, phage-alkyne). Dynamic covalent bonding chemistries include, but are not limited to, imine formation (polymer-aldehyde, phage-amine), hydrazone formation (polymer-hydrazine. (oxidized) phage-aldehyde), and oxime formation (polymer-hydroxylamine+(oxidized) phage-aldehyde).

In some embodiments, weak, dynamic covalent bonds are used to couple the bacteriophage to the hydrogel, so that the bacteriophage will release from the hydrogel over time due to unbinding of the link between the bacteriophage and the hydrogel followed by diffusion of the bacteriophage out of the hydrogel.

In some embodiments, combinatorial covalent crosslinking is used to couple the bacteriophage to the hydrogel with controlled hydrogel degradation. Hydrogel degradation mechanisms include: oxidation of polysaccharides such as alginate, chitosan, and hyaluronic acid for hydrolytic degradation: polyester hydrolysis for PCL, PLA, and PLGA: and microbial degradation, e.g., bacteria metabolization, for PVA.

In some embodiments, the hydrogel is formed from an oxidized alginate polymer activated with 1-ethyl-3-(3-dimethylamino) propyl carbodiimide (EDC) and N-hydroxysuccinimide (NHS), with calcium used as a crosslinker. Phage covalent bonding can be mediated via covalent carbodiimide chemistry (phage-amine). Degradation can be due to hydrolytic degradation of the oxidized alginate, with the timescale of degradation controlled by varying the degree of oxidation of the alginate.

In some embodiments, the hydrogel is formed from a hydrazine-coupled polymer (alginate, hyaluronic acid (HA), chitosan) and an aldehyde-coupled polymer (alginate, HA, chitosan), with crosslinking occurring from hydrazone bonding between the hydrazine and aldehyde. Phage covalent bonding can be mediated by imine formation (polymer-aldehyde+phage-NH₂). Degradation of the gel can be due to hydrolytic degradation of the oxidized polysaccharides.

In some embodiments, the hydrogel is formed from a norbornene-coupled polymer (oxidized alginate, HA, chitosan) and a tetrazine-coupled polymer (oxidized alginate, HA, chitosan) cross linked together via Click chemistry. Phage covalent bonding to the hydrogel can occur via thiol-ene chemistry. Degradation of the gel can be due to hydrolytic degradation of the oxidized polysaccharides.

In some embodiments, the hydrogel is formed from PVA-hydrazine cross linked with boric acid. Phage covalent bonding can occur via hydrazone formation (phage-aldehyde). Degradation can occur due to bacteria metabolization.

In some embodiments, the hydrogel is formed from collagen activated with EDC/NHS. Phage covalent bonding to the hydrogel can occur via carbodiimide chemistry (phage-NH₂). Degradation can occur due to hydrolytic degradation of polyester.

In some embodiments, the hydrogel is formed from PEG-acrylate, PEG-lysine, and PEG-glutamine crosslinked with thrombin-activated FXIIIa. Phage covalent bonding to the hydrogel can occur via carbodiimide chemistry (phage-COOH). Degradation can occur due to hydrolytic degradation of ester.

In some embodiments, the hydrogel is formed from PEG-VS (vinylsulfone) and PEG-diester-dithiol. Phage covalent bonding to the hydrogel can occur via vinylsulfone-thiol reaction. Degradation can occur due to hydrolytic degradation of ester.

In some embodiments, the hydrogel is formed from (polyethylene glycol)-co-(poly(hydroxy acid) diacrylate) with crosslinking via photoinitiation. Phage covalent bonding to the hydrogel can occur via acrylate-amine reaction. Degradation can occur due to hydrolytic degradation of α-hydroxy acid.

The provided hydrogel can be configured to have an average pore size suitable for allowing bacteriophage release from the gels over a desired timescale. In some embodiments, the average pore size configuration of the hydrogel is determined by the choice of crosslinking chemistry, e.g., linker agent type and crosslinking reaction parameters used to form the hydrogel. In some embodiments, methods of producing the provided hydrogel include selecting a crosslinking chemistry and crosslinking reaction parameters that will generate pores of a predetermined average size in the hydrogel. The average pore size of the hydrogel can be, for example, between 3 nm and 30 nm, e.g., between 3 nm and 11.9 nm, between 3.8 nm and 15 nm, between 4.8 nm and 18.9 nm, between 6 nm and 23.8 nm, or between 7.5 nm and 30 nm. In terms of upper limits, the hydrogel average pore size can be less than 30 nm, e.g., less than 23.8 nm, less than 18.9 nm, less than 15 nm, less than 11.9 nm, less than 9.5 nm, less than 7.5 nm, less than 6 nm, less than 4.8 nm, or less than 3.8 nm. In terms of lower limits, the hydrogel average pore size can be greater than 3 nm, e.g., greater than 3.8 nm, greater than 4.8 nm, greater than 6 nm, greater than 7.5 nm, greater than 9.5 nm, greater than 11.9 nm, greater than 15 nm, greater than 18.9 nm, or greater than 23.8 nm. Larger average pore sizes, e.g., greater than 30 nm, and smaller average pore sizes, e.g., less than 3 nm, are also contemplated.

In some embodiments, the provided hydrogel is prepared by selecting a suitable biopolymer, and contacting the biopolymer with one or more linking agents, i.e., crosslinkers, to form the hydrogel from the biopolymer. In some embodiments, the phages to be located within the interior of the hydrogel can be contacted with the biopolymer prior subsequent to the formation of the hydrogel from the biopolymer. In some embodiments, the phages can be contacted with the hydrogel subsequent to its formation. In some embodiments, the biopolymer comprises or consists of alginate. In some embodiments, the linking agents comprise or consist of multi-arm PEG-aldehyde and/or multi-arm PEG-benzaldehyde.

In some embodiments, the biopolymer is functionalized prior or subsequent to the hydrogel formation by contacting with a functionalization agent. In some embodiments, and as illustrated in FIG. 3, the functionalizing agent comprises or consists of azido-hydrazine. The contacting of the biopolymer or hydrogel with the functionalizing agent can be under conditions suitable and sufficient for functionalizing a desired portion of available reactive groups of the biopolymer or hydrogel. The portion of the available biopolymer or hydrogel reactive groups thus functionalized can be, for example, between 3% and 30%, e.g., between 3% and 11.9%, between 3.8% and 15%, between 4.8% and 18.9%, between 6% and 23.8%, or between 7.5% and 30%. In terms of upper limits, the functionalized portion of the reactive groups can be less than 30%, e.g., less than 23.8%, less than 18.9%, less than 15%, less than 11.9%, less than 9.5%, less than 7.5%, less than 6%, less than 4.8%, or less than 3.8%. In terms of lower limits, the functionalized portion of the reaction groups can be greater than 3%, e.g., greater than 3.8%, greater than 4.8%, greater than 6% greater than 7.5%, greater than 9.5%, greater than 11.9%, greater than 15%, greater than 18.9%, or greater than 23.8%. Larger portions, e.g., greater than 30%, and smaller portions, e.g., less than 3%, are also contemplated

The following embodiments are contemplated. All combinations of features and embodiment are contemplated.

Embodiment 1: A hydrogel comprising a plurality of bacteriophages located within the interior of the hydrogel, wherein each of at least a portion of the bacteriophages is connected to the hydrogel via one or more covalent bonds, and wherein the hydrogel is engineered to facilitate a sustained release of the connected bacteriophages.

Embodiment 2: An embodiment of embodiment 1, wherein the one or more covalent bonds comprise one or more dynamic covalent bonds.

Embodiment 3: An embodiment of embodiment 2, wherein the one or more covalent bonds comprise, bonds between lysine groups of the plurality of bacteriophages and aldehyde groups of the interior of the hydrogel.

Embodiment 4: An embodiment of embodiment 2, wherein the one or more covalent bonds comprise, bonds between aldehyde groups of the plurality of bacteriophages and hydrazine groups of the interior of the hydrogel.

Embodiment 5: An embodiment of any of the embodiments of embodiment 1-4, wherein the sustained release comprises cleavage of the one or more covalent bonds.

Embodiment 6: An embodiment of any of the embodiments of embodiment 1-5, wherein the sustained release comprises degradation of the hydrogel.

Embodiment 7: An embodiment of embodiment 6, wherein the degradation comprises oxidation of the hydrogel.

Embodiment 8: An embodiment of embodiment 6 or 7, wherein the degradation comprises microbial degradation.

Embodiment 9: An embodiment of any of the embodiments of embodiment 1-8, wherein the hydrogel comprises alginate.

Embodiment 10: An embodiment of any of the embodiments of embodiment 1-9, wherein the hydrogel has an average pore size between 3 nm and 30 nm.

Embodiment 11: A method of forming a hydrogel comprising a plurality of bacteriophages located within the interior of the hydrogel, the method comprising: selecting a biopolymer; contacting the biopolymer with a linking agent under conditions suitable for forming the hydrogel: and contacting the hydrogel with the plurality of bacteriophages under conditions suitable for binding at least a portion of the bacteriophages to the interior of the hydrogel.

Embodiment 12: An embodiment of embodiment 11, further comprising: contacting the biopolymer with a functionalizing agent under conditions suitable for functionalizing at least a portion of available reactive groups of the biopolymer.

Embodiment 13: An embodiment of embodiment 12, wherein the available reactive groups of the biopolymer comprise carboxylate groups.

Embodiment 14: An embodiment of embodiment 12 or 13, wherein the functionalizing agent comprises a hydrazine group.

Embodiment 15: An embodiment of any of the embodiments of embodiment 12-14, wherein the contacting of the biopolymer with the functionalizing agent occurs prior to the contacting of the biopolymer with the linking agent.

Embodiment 16: An embodiment of any of the embodiments of embodiment 12-15, wherein the contacting of the biopolymer with the functionalizing agent comprises functionalizing greater than 3% of the available reactive groups of the biopolymer.

Embodiment 17: An embodiment of any of the embodiments of embodiment 11-16, wherein the linking agent comprises one or more aldehyde groups.

Embodiment 18: An embodiment of any of the embodiments of embodiment 11-17, wherein the linking agent comprises a multi-arm linking agent.

Embodiment 19: An embodiment of any of the embodiments of embodiment 11-18, wherein the linking agent comprises polyethylene glycol (PEG).

Embodiment 20: An embodiment of any of the embodiments of embodiment 11-19, wherein the linking agent is selected from the group consisting of a PEG-aldehyde, a PEG-benzaldehyde, and a combination thereof.

Embodiment 21: An embodiment of any of the embodiments of embodiment 11-20, wherein the binding of at least a portion of the bacteriophages to the interior of the hydrogel comprises forming bonds between lysine groups of the plurality of bacteriophages and aldehyde groups of the interior of the hydrogel.

Embodiment 22: An embodiment of any of the embodiments of embodiment 11-21, wherein the binding of at least a portion of the bacteriophages to the interior of the hydrogel comprises forming bonds between aldehyde groups of the plurality of bacteriophages and hydrazine groups of the interior of the hydrogel.

Embodiment 23: An embodiment of any of the embodiments of embodiment 11-22, wherein the biopolymer comprises alginate.

Embodiment 24: An embodiment of any of the embodiments of embodiment 11-23, wherein the biopolymer has an average molecular weight between 10 kDa and 80 kDa.

Embodiment 25: An embodiment of any of the embodiments of embodiment 11-24, wherein the hydrogel has an average pore size between 3 nm and 30 nm.

Embodiment 26: A method for treating a patient suffering from a bacterial infection, the method comprising administering to the patient an effective amount of the hydrogel of an embodiment of any of the embodiments of embodiment 1-10.

Embodiment 27: An embodiment of embodiment 26, wherein the bacterial infection comprises an orthopaedic implant infection.

### Examples

The present disclosure will be better understood in view of the following non-limiting examples. The following examples are intended for illustrative purposes only and do not limit in any way the scope of the present invention.

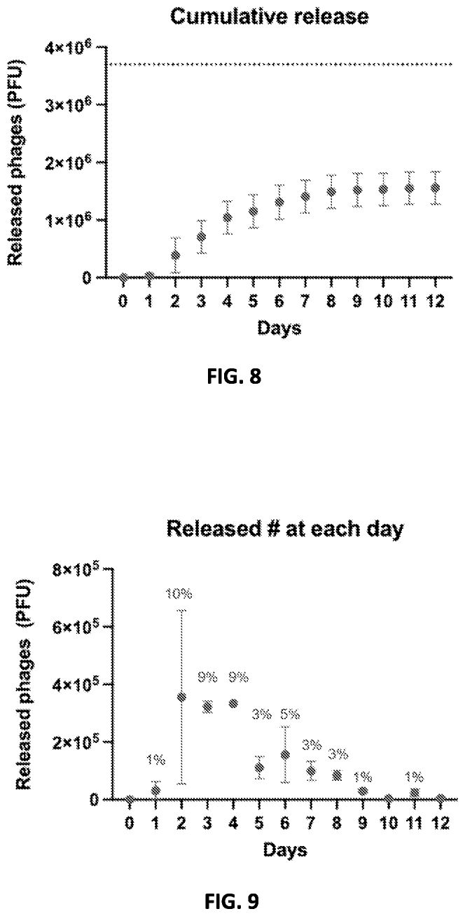

In one example, sustained release of bacteriophage from a hydrogel as disclosed herein has been demonstrated over a timescale of 1 week (FIGS. 8 and 9). The release was mediated by unbinding cleavage of dynamic covalent bonds between the bacteriophage and the hydrogel. The hydrogel was an alginate hydrogel formed by functionalizing the alginate with hydrazine as a functionalizing agent, and crosslinking the functionalized alginate with multi-arm PEG-aldehyde and PEG-benzaldehyde crosslinkers as linking agents. Phages bonded to the hydrogel via imine bonds between lysine groups of the phage and aldehyde PEG groups on the PEG crosslinkers and/or via aldehyde groups on the phage capsid binding to hydrazine groups on the alginate. Phage release was quantified using bacterial lawn assays. The release kinetics can be tuned by varying hydrogel properties. Additionally, this approach can be extended to other hydrogel systems by coupling aldehyde or hydrazine groups to such hydrogels thereby making them available for binding to phage.

Low molecular weight alginate (VLVG) (Pronova, UP VLVG, 28 kDa) was modified with alkyne, followed by the addition of azido-hydrazine (HYD) via Click reaction. The resulting VLVG-HYD was dialyzed against deionized water and then lyophilized. The degree of substitution of alginate was quantified using NMR spectroscopy, showing that 9.6% of the carboxylate group on alginate backbone have been functionalized with hydrazine.

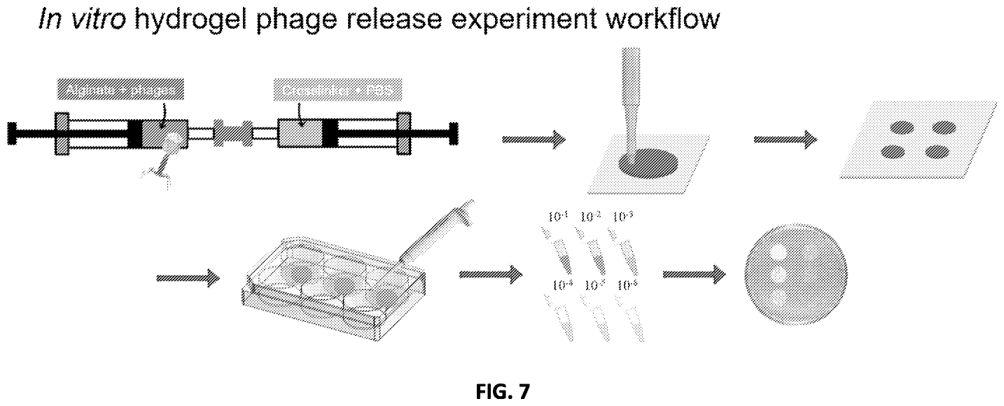

The modified alginate VLVG-HYD and multi-arms poly (ethylene glycol) (PEG) crosslinker with either aldehyde or benzaldehyde group (CreativePEGWorks or SENOPEG) were dissolved in Dulbecco's phosphate-buffered saline (DPBS) (modified with calcium and magnesium, HyClone). Hydrogels were formed by mixing above components through syringe and Luer lock along with desired LBL3 bacteriophage titer and addition of DPBS for remaining volume (FIG. 7). The mixture was then deposited between two glass plates spaced 2 mm apart. Subsequently, phage-hydrogels disks were punched out using a biopsy punch after 40 min of gelation.

As illustrated in FIG. 7, each hydrogel disk was plated in a well of 12-well non treated tissue culture plate (Falcon) and immersed in 1 mL of DPBS. The well-plate was left in incubator at 37° C. Supernatant containing released phages was collected every 24 hours and each hydrogel was moved to a new well containing fresh DPBS. To quantify the released phage titer, a spot assay of Pseudomonas aeruginosa AA245 lawn was infected with 3 uL of serial dilutions of collected supernatant.

The polymer backbone of the hydrogel is not limited to alginate. For example, other biopolymers including chitosan and hyaluronic acid can also be modified with hydrazine via Click chemistry. Those modified polymers will thus form similar dynamic crosslinked hydrogels with suitable linking agents, e.g., multi-arms PEG crosslinker with either aldehyde or benzaldehyde. The hydrogel could be formed with solely synthetic polymer as well. For example, mixing multi-arms PEG-hydrazine with multi-arms PEG-aldehyde or PEG-benzaldehyde will yield similar dynamic crosslinked hydrogels but with different microscopic crosslinking structure.

Although the foregoing disclosure has been described in some detail by way of illustration and example for purpose of clarity of understanding, one of skill in the art will appreciate that certain changes and modifications within the spirit and scope of the disclosure may be practiced, e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the disclosure. In addition, each reference provided herein is incorporated by reference in its entirety for all purposes to the same extent as if each reference was individually incorporated by reference.

1. A hydrogel comprising a plurality of bacteriophages located within the interior of the hydrogel, the hydrogel formed from one or more biopolymers, wherein each of at least a portion of the bacteriophages is connected to the hydrogel via one or more covalent bonds, and wherein the hydrogel is engineered to facilitate a sustained release of the connected bacteriophages.

2. The hydrogel of claim 1, wherein the one or more covalent bonds comprise one or more dynamic covalent bonds.

3. The hydrogel of claim 2, wherein the one or more covalent bonds comprise bonds between amine groups of the plurality of bacteriophages and aldehyde groups of the interior of the hydrogel.

4. The hydrogel of claim 2, wherein the one or more covalent bonds comprise bonds between aldehyde groups of the plurality of bacteriophages and hydrazine groups of the interior of the hydrogel.

5-8. (canceled)

9. The hydrogel of claim 1, wherein the one or more biopolymers comprise alginate.

10. The hydrogel of claim 1, wherein the hydrogel has an average pore size between 3 nm and 30 nm.

11. A method of forming a hydrogel comprising a plurality of bacteriophages located within the interior of the hydrogel, the method comprising:
selecting a biopolymer;
contacting the biopolymer with a linking agent under conditions suitable for forming the hydrogel; and
contacting the hydrogel with the plurality of bacteriophages under conditions suitable for binding at least a portion of the bacteriophages to the interior of the hydrogel.

12. The method of claim 11, further comprising:
contacting the biopolymer with a functionalizing agent under conditions suitable for functionalizing at least a portion of available reactive groups of the biopolymer.

13. The method of claim 12, wherein the available reactive groups of the biopolymer comprise carboxylate groups.

14. The method of claim 12, wherein the functionalizing agent comprises a hydrazine group.

15. The method of claim 12, wherein the contacting of the biopolymer with the functionalizing agent occurs prior to the contacting of the biopolymer with the linking agent.

16. (canceled)

17. The method of claim 11, wherein the linking agent comprises one or more aldehyde groups.

18. The method of claim 11, wherein the linking agent comprises a multi-arm linking agent.

19. The method of claim 11, wherein the linking agent comprises polyethylene glycol (PEG).

20. (canceled)

21. The method of claim 11, wherein the binding of at least a portion of the bacteriophages to the interior of the hydrogel comprises forming bonds between amine groups of the plurality of bacteriophages and aldehyde groups of the interior of the hydrogel.

22. The method of claim 11, wherein the binding of at least a portion of the bacteriophages to the interior of the hydrogel comprises forming bonds between aldehyde groups of the plurality of bacteriophages and hydrazine groups of the interior of the hydrogel.

23. The method of claim 11, wherein the biopolymer comprises alginate.

24. The method of claim 11, wherein the biopolymer has an average molecular weight between 10 kDa and 80 kDa.

25. The method of claim 11, wherein the hydrogel has an average pore size between 3 nm and 30 nm.

26. A method for treating a patient suffering from a bacterial infection, the method comprising administering to the patient an effective amount of the hydrogel of claim 1.

27. (canceled)
